(19) United States
(12) Patent Application Publication
(10) Pub. No.: US 2021/0125720 A1
(43) Pub. Date: Apr. 29, 2021

(54) HCC CODING NOTIFICATIONS

(71) Applicant: KONINKLIJKE PHILIPS N.V., EINDHOVEN (NL)

(72) Inventors: Chih-Wen CHENG, Alpharetta, GA (US); Bryant MENN, Alpharetta, GA (US); Vincent EMANUELE, Alpharetta, GA (US)

(21) Appl. No.: 16/494,324

(22) PCT Filed: Mar. 7, 2018
(86) PCT No.: PCT/EP2018/055526

§ 371 Date: Sep. 16, 2019

Related U.S. Application Data
(60) Provisional application No. 62/472,873, filed on Mar. 17, 2017.
(60) Provisional application No. 62/520,260, filed on Jun. 15, 2017.

(51) Int. Cl. G16H 50/20; G16H 10/60; G06N 5/00; G06N 20/00; G06F 17/16

(52) U.S. Cl. CPC G16H 50/20; G16H 10/60; G06F 17/16; G06N 20/00; G06N 5/003

(57) ABSTRACT

In one embodiment, a computer-implemented method, comprising: receiving information from plural patients, the information from each of the plural patients comprising one or more items recorded over a predefined period of time; formatting the items for use in a machine learning algorithm, wherein the formatting comprises: associating each of the one or more items with a respective word; forming a sentence for each of the plural patients from the words; and transforming the sentences for the plural patients into a numerical matrix, wherein the matrix comprises one row per patient and one column per word, each word weighted by occurrence; training models according to the machine learning algorithm based on the formatted items of the plural patients; determining if a first patient should have a hierarchical condition category code by applying one or more formatted items of the first patient to the trained models; and providing a notification of any suspect hierarchical condition category codes based on the application of the one or more formatted items of the first patient to the trained models.

### FIELD OF THE INVENTION

The present invention is generally related to medical diagnosis coding.

### BACKGROUND OF THE INVENTION

Medicare, which is administered by Centers for Medicare and Medicaid Services or CMS, is a federal program implemented in the United States to provide heath coverage for people 65 years of age or older and/or for people with certain qualified disabilities. Medicare may be grouped into one of four plans, including Parts A (for in-patient hospital coverage), B (for outpatient services and generally, services not covered by Part A), Part C (which includes Medicare Advantage, and provides Medicare benefits through capitated health insurance), and Part D (prescription drug). Focusing in particular on Medicare Advantage and reimbursement models, CMS relies on risk adjustment models to estimate the health cost expenditure, and reimburses Medicare Advantage plan providers based on demographics and health status of their enrollees. Risk adjustment data is typically extracted from diagnosis data reported from claims data and medical record documentation from physician offices, hospital impatient and outpatient settings. In particular, Medicare Advantage risk adjustment is based on what is known as Hierarchical Condition Category (HCC) codes. HCC codes, the quantity (e.g., seventy-nine (79) HCC codes in 2015) of which may vary from one year to another for reimbursement purposes, provide an important tool for mapping thousands of diagnosis codes (e.g., ICD-9 and 10 codes) to a reduced set of categories in the form of data that risk adjustment models can use to provide risk adjustment calculations or generally, risk scores (e.g., risk adjustment factor scores). The risk scores, in turn, are used to estimate reimbursement costs for the providers under the Medicare Advantage plan.

Each patient-based reimbursement is linked to the medical condition of the patient as documented by the diagnosis codes and demographics (e.g., gender, age, county, etc.). The risk scores are adjusted based on the documented diagnosis, which suggests the importance of the accuracy of the diagnosis. For instance, inaccurate diagnosis codes may result in inaccurate HCCs, which in turn may result in generally two types of inaccurate reimbursement payments: overpayment and underpayment. Overpayment refers to a situation where the Medicare Advantage plan provider submits codes containing unnecessary medical conditions, the corresponding coding referred to as over-coding. Plans with a higher chance of overpayments may expose the beneficiary of any reimbursements to a higher risk of penalties. Underpayment is the result of patient conditions that are not completely charted and hence the corresponding HCC codes are not submitted to CMS. The latter situation corresponds to missing codes. In underpayment instances, the reimbursed amount is less than the real expenses. Plans that have a disproportionally high share of underpayment enrollees may be at a competitive disadvantage.

A common solution for detecting and correcting over or missing codes is for clinical staff or a third party consultant to audit each patient's chart history. However, the success of this process relies on following experience-based rules and complicated database searches, a process that is prone to error and, generally, requires high costs and resources in terms of time and labor.

### SUMMARY OF THE INVENTION

In one embodiment, a computer-implemented method, comprising: receiving information from plural patients, the information from each of the plural patients comprising one or more items recorded over a predefined period of time; formatting the items for use in a machine learning algorithm, wherein the formatting comprises: associating each of the one or more items with a respective word; forming a sentence for each of the plural patients from the words; and transforming the sentences for the plural patients into a numerical matrix, wherein the matrix comprises one row per patient and one column per word, each word weighted by occurrence; training models according to the machine learning algorithm based on the formatted items of the plural patients; determining if a first patient should have a hierarchical condition category code by applying one or more formatted items of the first patient to the trained models; and providing a notification of any suspect hierarchical condition category codes based on the application of the one or more formatted items of the first patient to the trained models.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIGS. 1-3 are schematic diagrams that conceptually illustrate example processes by which an HCC coding notification system assesses patient information and notifies a provider about missing HCC codes or over-coding in accordance with an embodiment of the invention.

FIG. 4 is a schematic diagram that illustrates an example environment in which an HCC coding notification system is used in accordance with an embodiment of the invention.

FIG. 5 is a block diagram that illustrates an example server device in which an HCC coding notification system is implemented in accordance with an embodiment of the invention.

FIG. 6 is a schematic diagram that further illustrates preprocessing and training and validation processing in an HCC coding notification system in accordance with an embodiment of the invention.

FIGS. 7A-7B are schematic diagrams that conceptually illustrate model training in an HCC coding notification system in accordance with certain embodiments of the invention.

FIG. 8 is a screen diagram that illustrates an example user interface for providing a notification in an HCC coding notification system in accordance with an embodiment of the invention.

FIG. 9 is a flow diagram that illustrates an example method for notifying a user about the presence of over-coding and/or missing codes in an HCC coding notification system in accordance with an embodiment of the invention.

### DETAILED DESCRIPTION OF EMBODIMENTS

Disclosed herein are certain embodiments of an HCC coding notification system and method that detects the possibility of over-coding and missing codes associated with health services administered under Medicare Advantage health plans, and provides a notification of the presence of over-coding and missing codes for further review and possible correction by appropriate provider personnel or generally, a user (e.g., physician, physician assistant, lab tech, administrator, nurse, etc.). Herein, the codes associated with over-coding and missing codes collectively and individually are also referred to as suspect codes. In one embodiment, an HCC coding notification system receives patient domain data, transforms the data via a preprocessing component to enable machine learning and development of HCC models, and employs the machine learning for a given patient(s) to detect HCC suspect codes (e.g., HCC codes the patient should or should not have). A notification (e.g., recommendation, suggestion, flag, etc.) is provided directly or indirectly to a device of a health care provider and/or administrator involved with correctly documenting or using information from patient charts, prompting further investigation as to the need for further care or an update to a patient record (e.g., to correct the HCC coding via entry or deletion of a corresponding diagnosis code(s)).

Digressing briefly, patient care is documented under diagnosis codes (e.g., ICD-9 or 10 codes) that are ultimately segmented into HCC codes (e.g., using CMS tables) for purposes of risk evaluation and determination of reimbursement costs. The accuracy of such documentation is important, as missing codes may deprive health care providers of proper reimbursement and over-coding may result in unexpected, fiscally-challenging repayments for remedying over-compensation. Current approaches to ensuring the accuracy of HCC coding are manually intensive and/or deterministic (e.g., rules-based), and vary by the skill and/or experience level of the auditor. In contrast, certain embodiments of HCC coding notification systems automate the process over a larger data span not currently possible via manual audit without expending considerable time and resources (and revenue), and in particular, preprocess patient data to enable the use of machine learning techniques, use machine learning to train HCC models, and apply patient data to the HCC models to determine the presence (or absence) of suspect codes. Further, by providing notifications to the appropriate people, checks in the sufficiency of patient care may be made, as well as ensuring accurate HCC coding and proper reimbursement levels.

Having summarized various features of certain embodiments of an HCC coding notification system of the present disclosure, reference will now be made in detail to the detailed description of an HCC coding notification system as illustrated in the drawings. While the disclosure is described in connection with these drawings, there is no intent to limit it to the embodiment or embodiments disclosed herein. For instance, though HCC codes mapped from ICD-9 or -10 codes are described throughout, it should be appreciated by one having ordinary skill in the art in the context of the present disclosure that diagnostic codes other than ICD-9 or -10 codes may be used as a precursor to HCC codes, including SNOMED or any other industry-recognized diagnosis codes, or proprietary codes, as long as mapping to the corresponding HCC codes can be realized either directly or indirectly (e.g., mapping a SNOMED code to ICD-9 or -10 codes, which is then mapped to HCC codes). Further, although the description identifies or describes specifics of one or more embodiments, such specifics are not necessarily part of every embodiment, nor are all various stated advantages associated with a single embodiment. On the contrary, the intent is to cover all alternatives, modifications and equivalents included within the spirit and scope of an HCC coding notification system as defined by the appended claims. Further, it should be appreciated in the context of the present disclosure that the claims are not necessarily limited to the particular embodiments set out in the description.

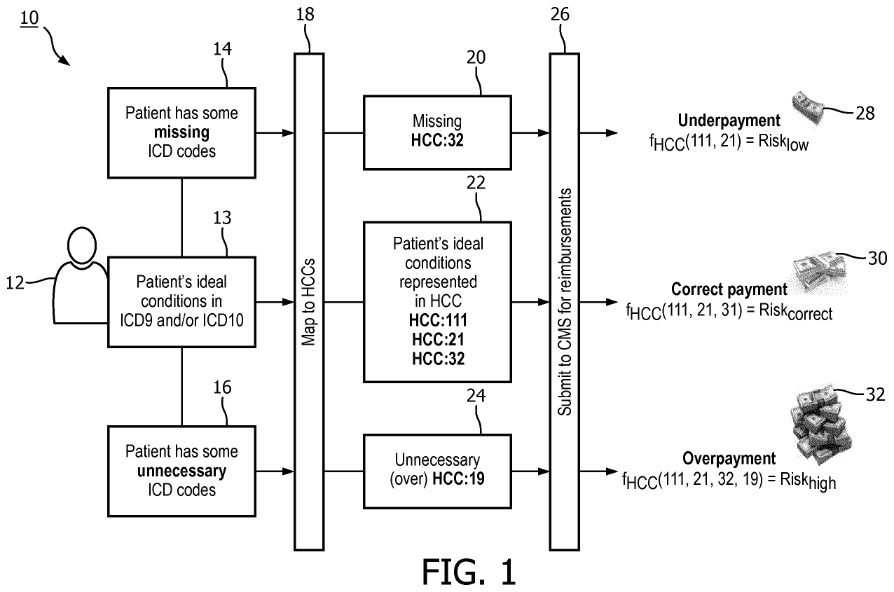

Attention is directed to FIGS. 1-3, which conceptually illustrate certain processes of an embodiment of an example HCC coding notification system. It should be appreciated by one having ordinary skill in the art in the context of the present disclosure that the processes presented in FIGS. 1-3 are illustrative of one example, and that variations to those depictions and corresponding descriptions may be employed and hence are contemplated to be within the scope of the disclosure. Referring to FIG. 1, an example process 10 is depicted with a representation of a patient 12 shown, the patient 12 having actual (true, ideal) conditions charted with ICD9 and/or ICD10 codes (generally also described as ICD codes hereinafter for brevity) as illustrated in block 13. However, illustrative of the types of problems certain embodiments of an HCC coding notification system seeks to address, the patient 12 may also have missing ICD codes as shown in block 14 and/or unnecessary ICD codes that are charted as shown in block 16. During the process 10, the patient information (e.g., patient diagnoses) is typically charted by a health care professional. Block 18 reveals that the ICD codes are mapped to HCC codes. In one embodiment, software in the HCC coding notification system (or in another system) may map the ICD codes to HCC codes using tables provided by CMS. Blocks 20 22, and 24 correspond respectively to missing HCC codes (e.g., HCC:32), codes for the actual or ideal conditions of the patient 12 (e.g., HCC:111, HCC:21, and HCC:32), and unnecessary codes or over-coding (e.g., HCC:19). In other words, the suspect HCC codes of blocks 20 and 24 may be the result of errors or omissions made in the ICD charting process. For instance, in the example depicted in FIG. 1, whereas the ideal or actual conditions shown in block 22 reveal HCC:32 among the correct HCC codes, a chart represented by block 20 may not include HCC:32 (and thus missing since the corresponding ICD codes was not charted). Also, where the ideal or actual conditions shown in block 22 include the HCC codes of HCC:111, HCC:21, and HCC:32 as the proper codes, during the charting process, an additional ICD code may have been included, which resulted in an unnecessary HCC code (e.g., HCC:19). When the HCC codes are submitted to CMS for reimbursement, as indicated in block 26, the missing HCC codes of block 20 comprising HCC codes HCC:111 and HCC:21 result in an underpayment (28) in view of the missing HCC:32, the correct HCC codes of HCC:111, HCC:21, and HCC:32 result in a correct payment (30), and the unnecessary HCC code of block 24 (HCC:19) incorrectly added to list of HCC codes HCC:111, HCC:21, and HCC:32 result in overpayment (32). It is the ideal condition that an embodiment of an HCC coding notification system seeks to reproduce.

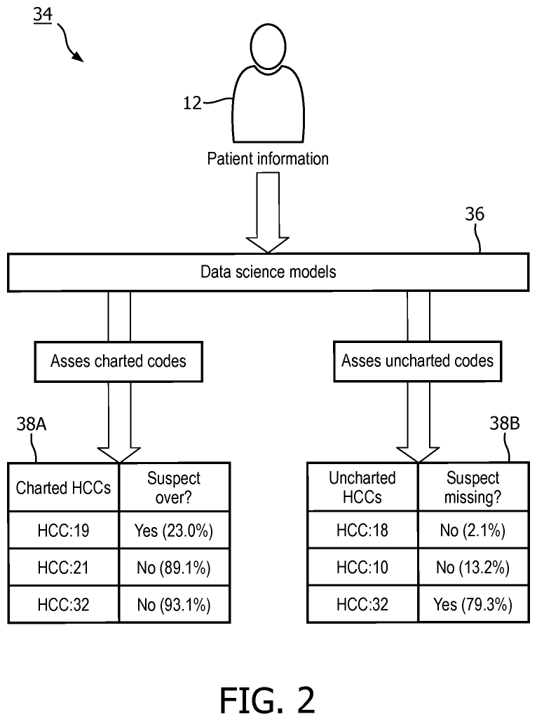

Having generally described the overall process 10 that may present suspect HCC codes for improper reimbursement for which certain embodiments of an HCC coding notification system seeks to address, attention is now directed to FIG. 2, which conceptually illustrates an example process 34 for notifying a health care professional or other user that is involved with the charting of ICD codes and/or the administration of HCC codes and/or reimbursement payments. In this example, assume from FIG. 1 the ideal or proper HCC codes of HCC:111, HCC:21, and HCC:32, respectively, and also the scenarios of FIG. 1 of missing codes and over-coding. The HCC coding notification system uses data science models 36 (herein, also data models) to evaluate the charted conditions of the patient 12. Responsive to application of the data models 36 are the provision of notifications 38. In one embodiment, notifications 38A and 38B for assessed charted codes and uncharted codes, respectively, each include the HCC codes, whether the HCC code is suspect (Yes) or not (No), and a confidence measure (e.g., here depicted as a percentage, but other measures may be used) that the patient 12 may actually have the HCC code. From the example in FIG. 2, the data models 36 determine a high percentage (e.g., 89.1% and 93.1%) for HCC codes HCC:21 and HCC:32, respectively. That is, there is a high probability that these codes are correct, and hence are not suggested as suspect codes (as designated “No” for each in the “suspect over?” column). On the other hand, HCC:19 is determined to have a low probability (e.g., 23.0%) of being a proper HCC code, and hence to the question of whether or not HCC:19 is over-coded, the answer is yes (i.e., HCC:19 is likely over-coded). After over-coding evaluation (or in some embodiments, concurrent with the over-coding assessment), an embodiment of the HCC coding notification system evaluates, using the data models 36, all HCC codes corresponding to the uncharted ICD codes (e.g., all less the three charted codes at present), one-by-one, for suspect missing codes. Each suggestion in the notification 38B, like in notification 38A, includes a confidence measure (e.g., percentage, though other measures may be used) and a Yes/No designation for the “suspect missing?” column. If the patient has a high probability of a condition corresponding to an uncharted ICD code (and hence, missing HCC code) and the data models 36 suggest a high percentage (e.g., HCC:32 at 79.3%), it is likely that the HCC:32 code is missing. On the other hand, if an HCC code corresponding to an uncharted ICD code has a low suggestion (e.g., HCC:18 at 2.1% and HCC:10 at 13.2%), the respective HCC codes are unlikely to be missing. Note that the thresholds for determining the confidence and what constitutes determining the recommendation is a yes or no is based on machine learning and varies based on the HCC code. This determination is based on the machine learning algorithm collecting data and making a decision as to the best threshold to use based on statistical analysis.

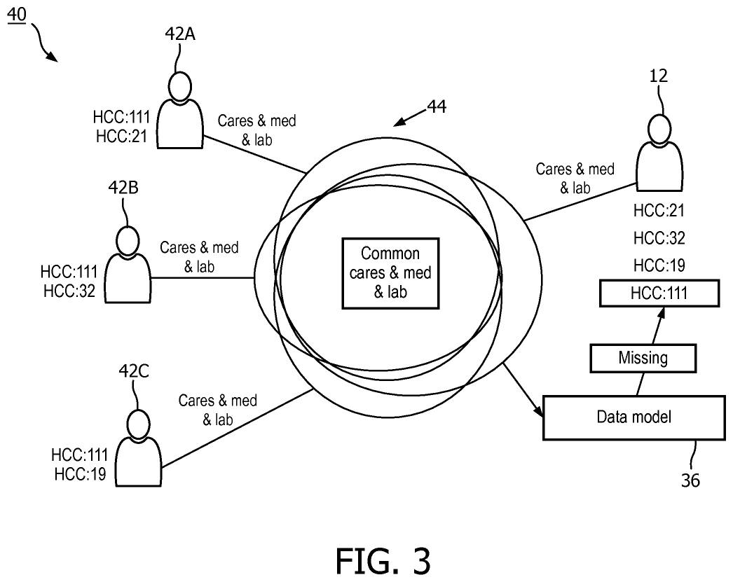

Referring now to FIG. 3, shown is a process 40 that illustrates, conceptually, a basis for the machine learning models (herein also referred to as data science models, data models, or HCC models) of certain embodiments of HCC coding notification systems. Shown are a population of patients 42 (e.g., 42A, 42B, and 42C) with respective patient information that may include any one or a combination of cares, medications, labs, or vitals (referred to herein also as items). Note that the items listed herein are not exhaustive, and that in some embodiments, additional and/or different items may be used that generally indicate a health or well-being of an individual, which may include demographic information. Items are designated in FIG. 3 as items 44. Cares refers to procedures, treatment, or generally, encounters between the health care profession and the patient. Medications include prescriptions and/or over-the counter medicines recommended by the health care professional. Labs include procedures that draw blood from or otherwise receive a sample from the patient. Vitals include direct or indirect measures of physiological conditions or parameters of the patient, including heart rate, blood pressure, blood sugar level, etc. Each circle or ellipse of the items 44 represents a set of medications, labs, cares (and in some embodiments, vitals) that belong to (e.g., are associated with) a patient 42. The overlap of circles is intended to convey the fact that there is a common set of items 44 amongst the patients with the given HCC codes. Stated otherwise, the common set of items 44 reveals that, for each of the patients 42 with their respective HCC codes (e.g., patient 42A with HCC:111 and HCC:21), patient 42B with HCC:111 and HCC:32, and patient 42C with HCC:111 and HCC:19), there is increased confidence that the common HCC code of HCC:111 should be included among those patients that have similar conditions. An underlying assumption of the data models 36 is that patients with similar conditions may share a certain level of features or information among patient charts (and hence share similar HCC codes). Based on this assumption, certain embodiments of an HCC coding notification system evaluates the HCC codes of the patients 42 by reviewing their chart items (e.g., cares, medications, labs). Three of the patients 42 share one common HCC code (HCC:111). That is, by comparing the charts, it is determined that there is a set of items 44 shared among these patients 42. Thus, when reviewing the items 44 of the patient 12 (who initially has HCC codes HCC:21, HCC:32, and HCC:19) and comparing to the items 44 of the patients 42 with common charts among each other and the patient 12, there is a high probability that patient 12 should have HCC:111. If HCC:111 is missing, the data models 36 detect this suspected coding inaccuracy and notify the provider or other relevant user or device of the possibility of the missing code to prompt further investigation and possibly correction according to the discretion of the provider (e.g., by charting an ICD code corresponding to HCC:111).

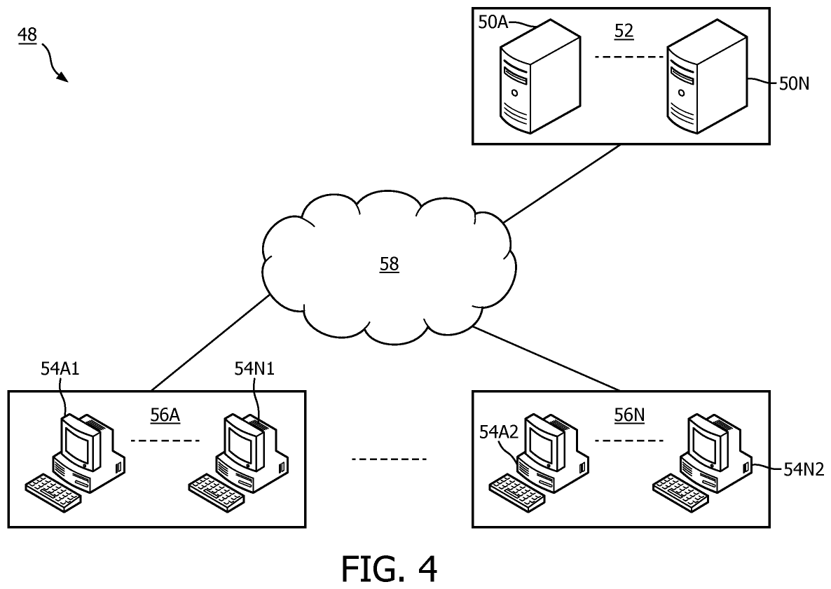

Having conceptually described operations of certain embodiments of an HCC coding notification system, attention is directed to FIG. 4, which illustrates an example environment 48 in which an HCC coding notification system is used in accordance with an embodiment of the invention. It should be appreciated by one having ordinary skill in the art in the context of the present disclosure that the environment 48 is one example among many, and that some embodiments of an HCC coding notification system may be used in environments with fewer, greater, and/or different components or system architectures than those depicted in FIG. 4. The environment 48 comprises a plurality of devices that enable communication of, or access to, information via one or more networks. The depicted environment 48 comprises one or more server devices 50 (e.g., 50A through 50N) of a server network 52 in communication with client devices 54 (e.g., 54A1 through 54N1, 54A2 through 54N2, etc.) of one or more health services facilities 56 (e.g., 56A through 56N) over one or more networks 58. For instance, the health services facilities 56 may include hospitals and ambulatory care facilities, and users may include physicians, nurse assistants, lab technicians, administrators, and any other users that chart patient information via the client devices 54.

The client devices 54 may include desktops, laptops, tablets, among other devices connected directly or indirectly to the network 58. The client devices 54 for each health services facility 56 may be coupled over a local area network (LAN) (e.g., a company intranet), including via wireless LAN (WLAN).

The network 58 may be any type(s) and/or form of network or networks, including a LAN, wide area network (WAN), including the Internet or World Wide Web, a metropolitan area network (MAN), public, private, etc. The network 58 may be comprised of devices interconnected over wireless or wired connections, or a combination of wired and wireless connections. For instance, the network 58 may include any one or combination of the following: a point to point network, a broadcast network, a wide area network, a local area network, a telecommunications network, a data communication network, a computer network, an ATM (Asynchronous Transfer Mode) network, a SONET (Synchronous Optical Network) network, a SDH (Synchronous Digital Hierarchy) network, a wireless network and a wireline network. In some embodiments, the network 58 may comprise a wireless link, such as an infrared channel or satellite band. The topology of the network 58 may be a bus, star, or ring network topology. The network 58 and network topology may be of any such network or network topology as known to those ordinarily skilled in the art capable of supporting the operations described herein.

In one embodiment, the server devices 50 of the server network 52 may provide cloud computing services, including remote data storage, data modeling applications, internet services, security services, content distribution, etc. to the client devices 54 of the health services facilities 56. In some embodiments, the server devices 50 may be coupled via a LAN (or wireless LAN, etc.), or coupled according to geographically separate facilities via the network 58 or other networks, such as to implement plural cloud systems. When embodied as a cloud service or services, the server devices 50 may comprise an internal cloud, an external cloud, a private cloud, or a public cloud (e.g., commercial cloud). For instance, a private cloud may be implemented using a variety of cloud systems including, for example, Eucalyptus Systems, VMWare vSphere®, or Microsoft® HyperV. A public cloud may include, for example, Amazon EC2®, Amazon Web Services®, Terremark®, Savvis®, or GoGrid®. Cloud-computing resources provided by these clouds may include, for example, storage resources (e.g., Storage Area Network (SAN), Network File System (NFS), and Amazon S3®), network resources (e.g., firewall, load-balancer, and proxy server), internal private resources, external private resources, secure public resources, infrastructure-as-a-services (IaaSs), platform-as-a-services (PaaSs), or software-as-a-services (SaaSs). The cloud architecture of the server devices 50 may be embodied according to one of a plurality of different configurations. For instance, if configured according to MICROSOFT AZURE™, roles are provided, which are discrete scalable components built with managed code. Worker roles are for generalized development, and may perform background processing for a web role. Web roles provide a web server and listen and respond for web requests via an HTTP (hypertext transfer protocol) or HTTPS (HTTP secure) endpoint. VM roles are instantiated according to tenant defined configurations (e.g., resources, guest operating system). Operating system and VM updates are managed by the cloud. A web role and a worker role run in a VM role, which is a virtual machine under the control of the tenant. Storage and SQL services are available to be used by the roles. As with other clouds, the hardware and software environment or platform, including scaling, load balancing, etc., are handled by the cloud.

In some embodiments, the server devices 50 may be configured into multiple, logically-grouped servers, referred to as a server farm. The server devices 50 may be geographically dispersed, administered as a single entity, or distributed among a plurality of server farms, executing one or more applications on behalf of one or more of the client devices 54. The server devices 50 within each farm may be heterogeneous. One or more of the server devices 50 may operate according to one type of operating system platform (e.g., WINDOWS NT, manufactured by Microsoft Corp. of Redmond, Wash.), while one or more of the other server devices 50 may operate according to another type of operating system platform (e.g., Unix or Linux). The group of server devices 50 may be logically grouped as a farm that may be interconnected using a wide-area network (WAN) connection or medium-area network (MAN) connection. The server devices 50 may each be referred to as a file server device, application server device, web server device, proxy server device, or gateway server device.

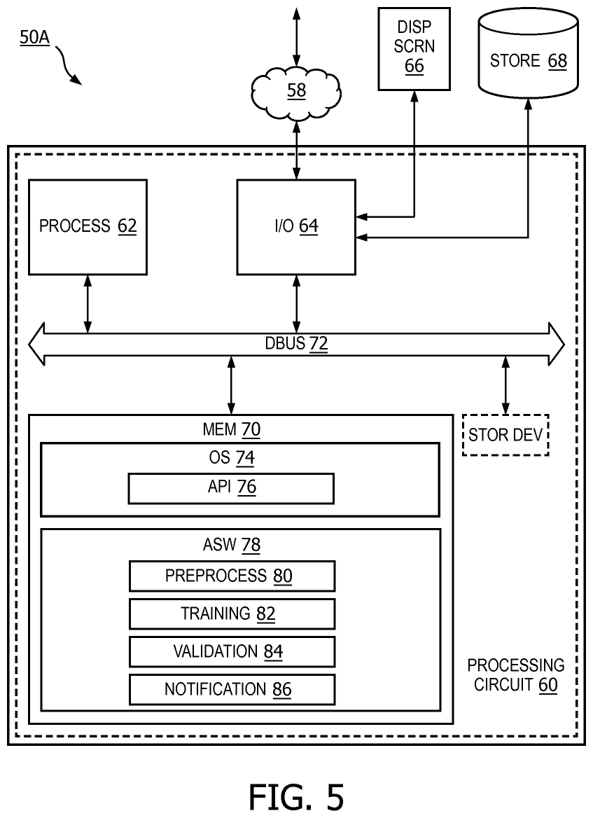

Referring now to FIG. 5, shown is an example server device 50A that may be used to detect suspect HCC codes and generate notifications. The server device 50A may be embodied as an application server device, and is also generally referred to herein as an apparatus. One having ordinary skill in the art should appreciate in the context of the present disclosure that the example server device 50A is merely illustrative of one embodiment, and that some embodiments of server devices may comprise fewer or additional components, and/or some of the functionality associated with the various components depicted in FIG. 5 may be combined, or further distributed among additional modules or devices, in some embodiments. The server device 50A is depicted in this example as a computer system, such as one providing a function of an application server device. It should be appreciated that certain well-known components of computer systems are omitted here to avoid obfuscating relevant features of the server device 50A. In one embodiment, the server device 50A comprises a processing circuit 60 (PROCES CKT 60) that comprises hardware, software, or a combination of hardware and software. In some embodiments, the processing circuit 60 may comprise a greater number or fewer components than those depicted in FIG. 5. In one embodiment, the processing circuit 60 comprises one or more processors, such as processor 62 (PROCES 62), input/output (I/O) interface(s) 64 (I/O 64), which in one embodiment is coupled to a display screen 66 (DISP SCRN 66) and one or more data storage devices, including data storage device 68 (STORE 68), and other user interfaces (e.g., keyboard, mouse, microphone, etc.), and memory 70 (MEM 70), all coupled to one or more data busses, including data bus 72 (DBUS 72). In some embodiments, the display screen 66, data storage device 68, and/or other user interfaces may be coupled directly to the data bus 72, or coupled to the server device 50A over the network 58. The memory 70 may include any one or a combination of volatile memory elements (e.g., random-access memory RAM, such as DRAM, and SRAM, etc.) and nonvolatile memory elements (e.g., ROM, Flash, solid state, EPROM, EEPROM, hard drive, tape, CDROM, etc.). The memory 70 may store a native operating system, one or more native applications, emulation systems, or emulated applications for any of a variety of operating systems and/or emulated hardware platforms, emulated operating systems, etc. In some embodiments, a separate data storage device may be coupled as a network-connected device (or devices) via the I/O interfaces 64 and the network 58. The data storage device 68 may be embodied as persistent memory (e.g., optical, magnetic, and/or semiconductor memory and associated drives) to store patient data (e.g., including cares, medicine, labs, vitals, or collectively, items, diagnosis codes, HCC codes, patient identifiers (IDs), demographics, etc.). In one embodiment, the data storage device 68 may operate according to SQL processing, though in some embodiments, the processing of the data storage device 68 may be based on non-SQL processing. Further, though a single storage device 68 is illustrated, in some embodiments, multiple of such data storage devices 68 (or other configurations) may be used. The data may be configured according to a data base structure, though other data structures may be used.

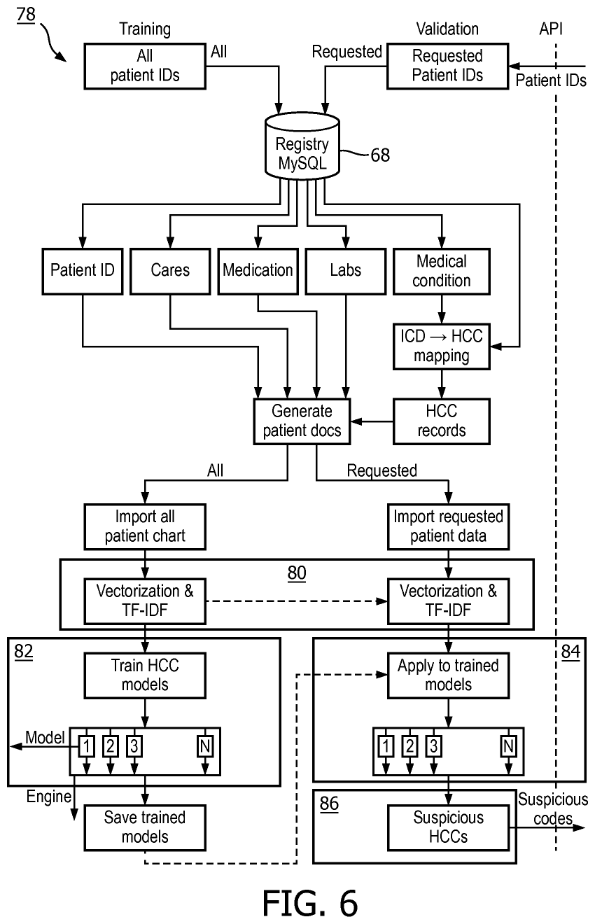

In the embodiment depicted in FIG. 5, the memory 70 comprises an operating system 74 (OS), which includes an application programming interface 76 (API 76). In one embodiment, the API 76 may enable the importation of patient information (e.g., patient ID) from another device (e.g., server device) to the storage device 68 and/or the communication of notifications to another device (e.g., server device), the latter which may be used to present the notifications to a user or communicate the notifications to a user device for presentation. The memory 70 further comprises application software 78 (ASW), which in one embodiment includes a preprocessing module 80 (PREPROCESS 80), a training module 82 (TRAINING 82), a validation module 84 (VALIDATION 84), and a notification module 86 (NOTIFICATION 86). In some embodiments, additional or fewer (e.g., combined functionality) modules to provide similar functionality may be used, either co-located in memory 78 (or storage device (STOR DEV)) or distributed among additional devices. As noted in conjunction with FIG. 4, functionality of the HCC coding notification system may be distributed among plural devices. In one embodiment, the functionality of the application software 78 may be distributed in scaled fashion among the server devices 50A-50N. For instance, preprocessing functionality may be performed in server device 50A, training functionality may be performed in server device 50B, validation functionality may be performed in server device 50C, and notification functionality may be performed in server device 50D. In some embodiments, the application software functionality may be distributed among fewer devices or additional devices than those described in the immediately prior example. It is assumed for purposes of illustration that the application software 78 resides entirely at the server device 50A, with the understanding that the various functional modules of the application software 78 may reside in additional devices. The application software 78 provides multiple functionality, including the importation of data of all patients from one or more customers (e.g., health care providers), vector space transformations (e.g., vectorization and TF-IDF), training of data models for each HCC code (e.g., in one embodiment, a data model per HCC code is provided), the storing of the data models (e.g., in memory 78, or in some embodiments, data storage device 68), the application of the data models to formatted (e.g., reused transformations) patient data, and the provision of notifications based on the results of the application of the data models. As explained further below, the preprocessing module 80 prepares patient information for use by the training module 82 and validation module 84. The preprocessing module 80 receives patient information from the data storage device 68, and applies vectorization and TF-IDF transformations to prepare the data for data model training and application to the data models by the training module 82 and validation module 84, respectively. The training module 82 and validation module 84 comprise the data (science) models 36 (FIG. 1) described above. The training module 82 uses machine learning (as opposed to deterministic processing) to train the data science models, and in one embodiment, may be scheduled to run at periodic intervals (e.g., once per week), aperiodically (e.g., in real-time), including based on one or more events (e.g., upload of new patient data, by request, etc.). The validation module 84, which performs operations periodically or aperiodically (e.g., in real-time, based on events, etc.), applies the data models to (formatted, or rather, transformed) patient information to derive one or more notifications. The notification module 86 receives the notification from the validation module 84, and formats and presents the recommendations (including a confidence measure in one embodiment) for delivery to a device for presentation (or communication to another device for presentation) to a user. In one embodiment, the notification module 86 uses the API 76 to communicate the notification to another device that operates in, for instance, a server-client relationship with client devices to present the notifications. In some embodiments, the notification module 86 may be a web server application that presents the notifications via a web-based user interface. The notifications may comprise the suspect HCC codes, recommendation (e.g., Yes or No as illustrated in FIG. 2) and a confidence measure (e.g., the percentage or probability as illustrated in FIG. 2), or other information that may be used at another device to alert the user that one or more HCC codes are suspect. For instance, in one embodiment, a user interface may be presented that lists the HCC codes for a given patient and distinguishes (e.g., flags, such as via color difference, warning icons, etc.) the suspect codes. Further description of the application software 78 and its component modules and the API 76 are described below in conjunction with FIG. 6.

Execution of the application software 78 may be implemented by the processor 62 under the management and/or control of the operating system 74. The processor 62 may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the server device 50A.

The I/O interfaces 64 comprise hardware and/or software to provide one or more interfaces to the network(s) 58, as well as to other devices such as the display screen 66, the data storage device 68, and other user interfaces. In other words, the I/O interfaces 64 may comprise any number of interfaces for the input and output of signals (e.g., analog or digital data) for conveyance of information (e.g., data) over various networks and according to various protocols and/or standards. The user interfaces may include a keyboard, mouse, microphone, immersive head set, etc., which enable input and/or output by an administrator or other user.

When certain embodiments of the server device 50A are implemented at least in part with software (including firmware), as depicted in FIG. 5, it should be noted that the software (e.g., including the application software 78 and its component modules) can be stored on a variety of non-transitory computer-readable medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the server device 50A are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

With continued reference to FIG. 5, attention is now directed to FIG. 6, which provides further functional detail of the application software 78. It should be appreciated by one having ordinary skill in the art that the functionality illustrated in the schematic diagram of FIG. 6 is one example, and that variations to the diagram may be made to achieve similar effect in functionality. The application software 78 comprises the preprocessing module 80, the training module 82, the validation module 84, and the notification module 86. Note that the component functionality shown in FIG. 6 above the preprocessing module 80 may be performed in other software or devices in some embodiments. As depicted in FIG. 6, patient information is imported to a MySQL registry, which in one embodiment may be embodied as the data storage device 68. As indicated above, though described using a SQL process, the data storage device 68 may also operate according to non-SQL technology in some embodiments. Patient information from among a wide population of patients (shown as all patient IDs) may be stored in the MySQL registry 68 and used in training data models, whereas a patient record with HCC codes evaluated for suspect HCC coding may be received for purposes of the application (validation) of the data models. From the MySQL registry 68, items (e.g., cares, medications, labs, etc.) and other patient information is extracted, and the ICD codes are mapped to HCC codes according to CMS tables. The preprocessing module 80 receives the imported patient information (including the items) from the MySQL registry 68 (or in some embodiments, from a plurality of data structures). MySQL is an open-source relational database management system that can be run as a virtual machine image or service on a cloud computing platform, including Amazon EC2, among other third party or proprietary platforms. The preprocessing module 80 may use SQL communications (e.g., GET, etc.) to access patient information from the MySQL registry 68. Note that the use of MySQL is one example embodiment, and that other relational database management system, including non-SQL data storage processing may be employed in some embodiments. The MySQL registry 68 stores patient information, including patient ID, items (e.g., cares, medications, labs), and medical conditions, and the diagnosis codes (e.g., ICD codes) are then mapped to HCC codes.

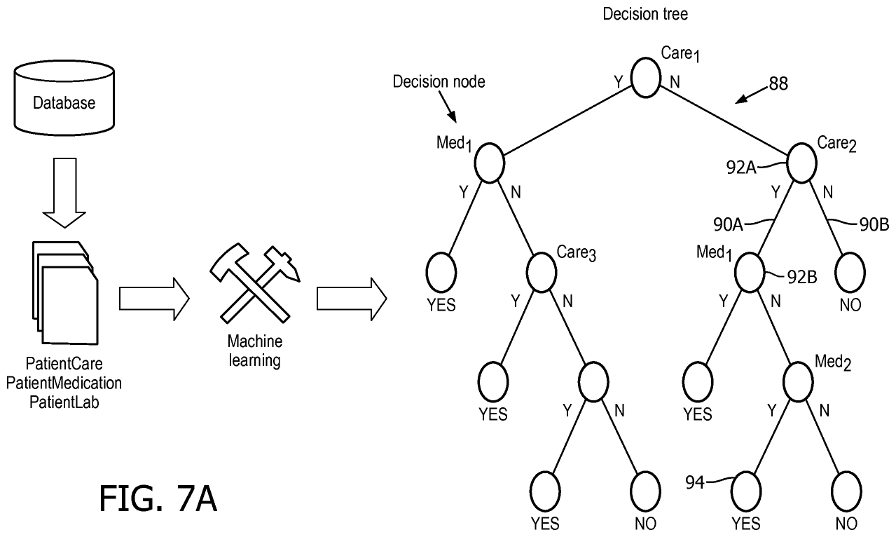

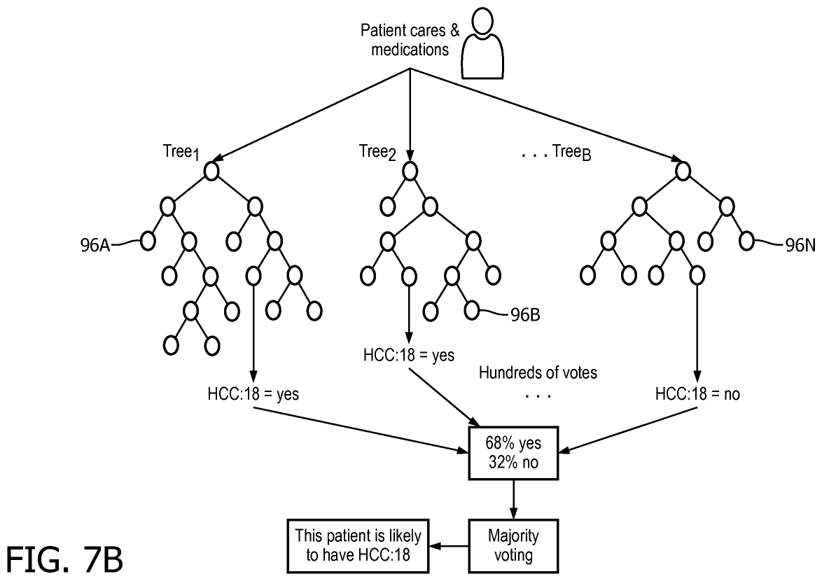

The preprocessing module 80 accesses the patient information, including the items for each patient, for all available patients from the MySQL registry 68. The preprocessing module 80 formats the data by performing vector space transformation on the patient data (information), using for instance, vectorization and TF-IDF transformations. Note that the items for each patient over a defined time interval may be used by the preprocessing module 80. For instance, items over a two (2) year window may be used, though other periods of time may be used. Given a set of items from each patient, the preprocessing module 80 quantitatively evaluates how important each item is and provides higher weights for the important ones, yet uses all of the items (regardless of weight) to train the data models (e.g., HCC models). In one embodiment, the preprocessing module 80 uses natural language processing techniques to apply the different weights to the items and to transform the items into a numerical format that can be used by the training module 82 to train the data models. For instance, each item is treated as a word in the form of a DictionaryName:Code (e.g., RxNorm:746719). Each patient is associated with a sentence consisting of words separated by white-spaces (e.g., CPT94305 RxNorm:746719 WC_lab:165). As expected, a patient with more illnesses may have a very long sentence and a healthy individual may have an empty or short sentence. Sentences from all patients are referred to herein as raw data. The preprocessing module 80 transforms the raw data into numerical feature vectors with a fixed size, which are derived from the raw text documents of variable length. In one embodiment, the preprocessing module 80 extracts numerical features from the text by tokenizing, counting, and normalizing. That is, the preprocessing module 80 tokenizes sentences with white spaces as word separators, and counts the occurrences of words in each sentence. The preprocessing module 80 normalizes and weights (e.g., with diminished importance) words that occur in the majority of patients. Note that in some embodiments, other word separators may be used, including commas, spaces, some ASCII characters that are presently used as field separators or any character not intended as a word. Afterwards, all patient sentences are represented by a large numerical matrix with one row per patient and one column per item (e.g., word) with weighted importance. In other words, the preprocessing module 80 uses the aforementioned process to convert the textual representation of patient information into a vector space model (VSM). The matrix is now ready for data model training performed by the training module 82. In model training, each model is trained based on a specific HCC code. For example, in embodiments that use 2015 CMS-based HCC coding (e.g., 79 HCC codes), there are 79 HCC models for the HCC training module 82. To further explain model training and validation, reference is made to FIGS. 7A-7B.

FIG. 7A conceptually illustrates data model training in a single decision tree, and FIG. 7B extends the description of data model training to multiple independent decision trees and illustrates an example of validation. Referring to FIG. 7A, when trying to determine if a patient should have a given HCC code, the chart items are examined carefully. Decision trees are a machine learning technique that are applied to the patient data formatted in vector space for plural patients. A decision tree 88 is shown in FIG. 7A, and comprises flowchart graphs or diagrams that are used to examine all of the decision alternatives and possible final outcomes (e.g., HCC=yes, or HCC=No, from FIG. 3). Model training involves training the decision tree 88 by using all patient items after vector space transformation (e.g., cares, labs, medication). Each branch, such as branch 90 (e.g., 90A or 90B, among other branches) of the decision tree 88, represents one of the possible options that are available when making a decision at a node, such as node 92 (e.g., 92A). The branches 90 can be extended when one alternative outcome leads to another decision (e.g., 92B from 90A) that must be made. Nodes appearing more to the top of the decision tree 88 are more important than those close to the bottom of the decision tree 88. In the depicted example shown in FIG. 7A, the item labeled, Care₁, is the most important item, and the item labeled, Med₁, is more important than the item labeled, Care₃. The decision tree growing process is completed with leafs, such as leaf 94, as final output decisions. The decision tree 88 is built from the items of a large pool of patients. The formatted (vector space transformed) items of a particular patient may be passed through the branches 90 and nodes 92 of the decision tree 88 during validation to determine what the final leafs 94 suggest. For instance, with a patient having Care₂ and Meds₂, the decision path would be Care₁=No (N) Care₂=Yes (Y), Med₁=N, Med₂=Y, and so HCC=Yes. In other words, the recommendation is that the patient needs to have the given HCC charted.

The decision tree processing performed by the training module 82 (for building the models) and the validation module 84 (for assessing whether a given HCC is suspect) can be improved upon. For instance, making decisions on all patients using only a single decision tree 88 is somewhat like relying on a single physician to make all diagnoses for all patients in a hospital. In other words, machine learning, like humans, may make decisions with a certain level of subjective bias. One mechanism to improve the process is for the training module 82 to build decision trees that are independent (or as independent as possible) of one another, which are used collectively as an ensemble to make the final decision. Such a process is referred to as ensemble learning, and in one embodiment, one particular technique used by the training module 82 to implement ensemble learning is referred to as random forest. Referring to FIG. 7B, random forest is considered a supervised data science technique, and generally involves building a plurality of decision trees 96 (e.g., 96A, 96B, . . . 96N). Randomness is inserted when building the decision trees 96 to make the decision trees 96 as independent relative to each other as possible. In one embodiment, randomness may be achieved by building each tree 96 based on a random subset of the training data set. In another embodiment, randomness may be achieved by building the trees 96 using a small subset of the available input variables and their corresponding values. When the validation module 84 is activated, to determine if a patient has the proper HCC codes, the validation module 84 passes the formatted patient data (e.g., items) to all of the decision trees 96, and each decision tree (e.g., 96A, 96B, . . . 96N) provides a decision of either HCC=Yes or HCC=No (e.g., for HCC:18). The decisions from each of the trees 96 can be tallied, providing in a sense, votes (e.g., a recommendation and confidence measure (e.g., percentage)) for the decisions (e.g., 68% yes, 32% no). Random forest chooses the decision having the most votes. Additional information regarding the use of random forests may be found in the publication, “Random Forest Classifier Combined with Feature Selection for Breast Cancer Diagnosis and Prognostic,” in J. Biomedical Science and Engineering, by Cuong Nguyen, Yong Wong, and Ha Nam Nguyen, published on-line in May 2013, incorporated herein by reference.

Referring back to FIG. 6, the preprocessing module 80 passes the results of vectorization and TF-IDF (e.g., re-uses the vectorization and TF-IDF transformers) determined from the training pipeline (based on a large population of patients) to the vectorization and TF-IDF component of the validation pipeline, and also saves the trained data models (the random forest decision trees 96). The trained data models are accessed by the validation module 84 and items (in a matrix) that have been formatted (by the passed transformers) via the preprocessing module 80 for the validation pipeline are passed through the plural decision trees to determine suspect HCC codes (or validate existing HCC codes). The validation module 84 is also referred to herein as an HCC validation engine, and comprises an ensemble of models built by the training module 82. In the recommendation stage, patient information for a particular patient is fed into the HCC validation engine, which passes the data to all 79 (e.g., using year 2015) HCC models developed in the training or building phase. Each model outputs its validated HCC result, and the engine collects from all of the models and outputs the validation results for all HCCs.

It should be appreciated by one having ordinary skill in the art that variations to the architecture and/or functionality of FIG. 6 are contemplated to be within the scope of the disclosure. For instance, though shown with vectorization outside of the models, in some embodiments, vectorization may be achieved on a per model basis (e.g., precomputed based on updated patient data, including before actual requests for patient updates).

The notification module 86 receives the decisions from the random forest processing or data models and formats for presentation the notifications regarding suspect or validated codes. In one embodiment, the notifications module 86 may communicate the notifications to another device or software entity via the API 76. In other words, the generation of the application software 78 may be segregated among different developers or distributed among different devices or virtual machines, and the API 76 may enable the notifications formatted by the notifications module 84 to be communicated to another component for presentation to the user. Thus, the API 76 enables the receipt of patient IDs by the application software 78, and enables the delivery of the HCC results for each patient from the notifications module 86 for presentation via another device. In one embodiment, the suggestions are in the form of [{code}, conf] with three possible codes: “-:” corresponding to no suggestion, “m:” corresponding to suggest missing, and “o:” corresponding to suggest over. “Conf” is a confidence measure (e.g., percentage) indicating that from all similar patients, what is the confidence or percentage that this HCC is from a suggested charted ICD code. That is to say, a high “conf” implies that the particular patient has a high chance to have the ICD code for the corresponding HCC code charted, and vice versa. For each HCC code, there is a threshold (learned from the machine learning process described above) to determine at what level of “conf” should it be suggested that the HCC needs to have a corresponding ICD code charted or not. For example, the HCC suggestion engine (e.g., the validation module 84) may serve two HCC codes: HCC:19 (diabetes without chronic complications) and HCC:18 (diabetes with chronic complications). The API request and response for, say three patients of a given customer (provider XYZ, which is a member of the applicationID in the API query below) may look like the following:
>>curl-X POST-d ‘{“patients”: [1284635, 1081671, 253962], “applicationID”:55}’
http://hcc.service.wellcentive.com
{“253962”: {“19”: [“-”, 82.31], “18”: [“m”, 76.52]}, “1284635”: {“19”: [“-”, 12.13], “18”: [“-”, 9.42]}, “1081671”: {“19”: [“-”, 64.32], “18”: [“o”, 23.41]}}

Note that there are generally many patient IDs associated with a single applicationID. In this example, the request corresponds to three patients: 1284635, 1081671, and 253962, which all belong to the applicationID of 55. Thus, in one embodiment, both the applicationID and the patient ID are specified to identify the patient. Explaining the above example further, the host server is identified generically with the serverID/service path or API server URL, hcc.service.wellcentive.com, and it should be appreciated that the location/identity of the server may be for one or more locations and/or identifications in some embodiments. Also shown is a POST command for customer XYZ (which is “55” in this example), and in particular for the requested patientID, 253962 (HCC19 charted, HCC18 uncharted), 1284635 (HCC19 uncharted, HCC18 uncharted), and 1081671 (HCC19 charted, HCC18 charted). Though described using a POST command, it should be appreciated by one having ordinary skill in the art in the context of the present disclosure that, in some embodiments, any form of network communications and/or protocols may be used.

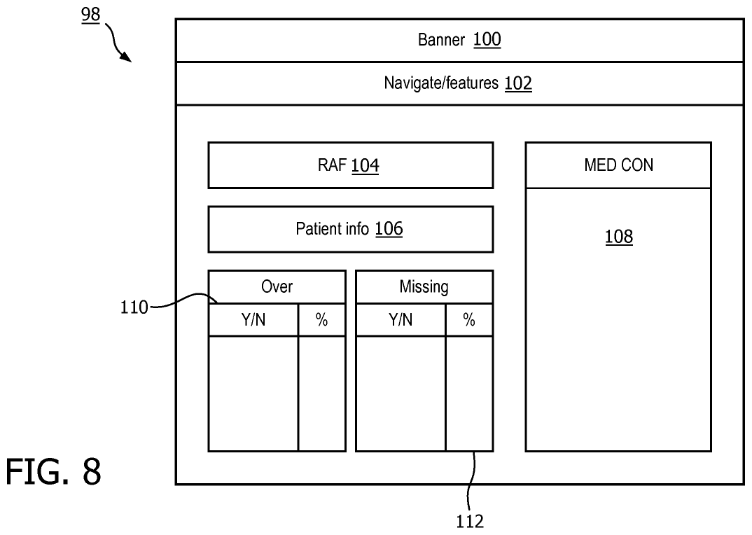

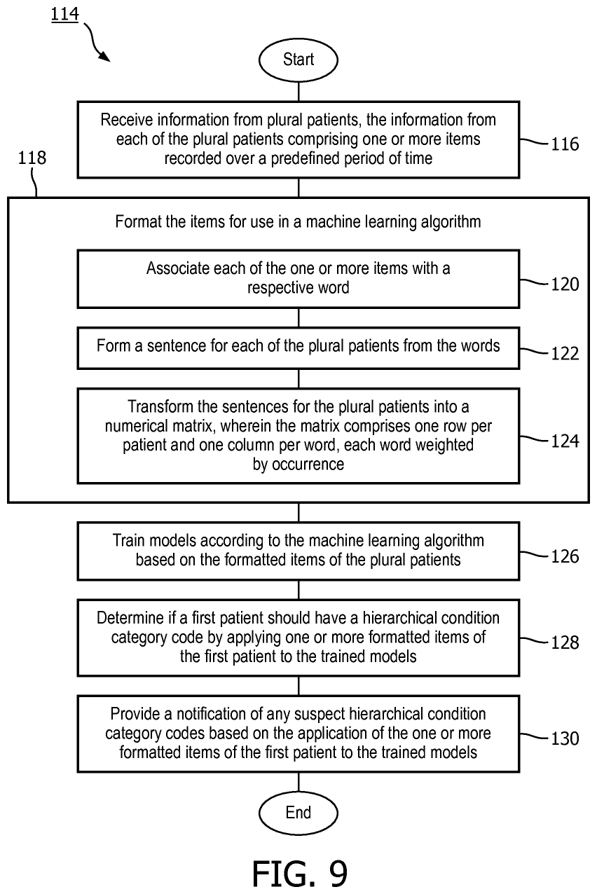

Referring to FIG. 8, shown is one example graphical user interface (GUI) 98 that may be provided by the notification module 96 (or for which content is provided via the API 76 for presentation by another device) to alert of suspect codes for a given patient based on the processing by the application software 78. It should be appreciated by one having ordinary skill in the art that the GUI 98 illustrates one example, and that variations to the design and/or content may be used in some embodiments. For instance, the GUI 98 may omit certain features or merely provide a flag of the suspect codes for a given patient. In some embodiments, the features presented in the GUI 98 of FIG. 8 may be presented over plural GUIs that may each be presented as a consequence of selections made in the prior presented GUI. The GUI 98 comprises a software source/service or provider identifying banner 100, a navigation or features option bar 102 comprising plural selectable features to prompt searches of patient information, charts, among other options, a risk adjustment factor (RAF) score field 104, a patient information field 106, a medical conditions (MED CON) field 108, and an over-coding (OVER) field 110 and missing code (MISSING) field 112. Additional or fewer fields may be included in the GUI 98 in some embodiments. The patient information field 106 may include the patient name, age, date of birth, as well as other information in some embodiments. The risk adjustment factor score field 104 is the cumulative score that is based on the patient demographics (e.g., age band, gender, etc.) and medical condition (e.g., ICD and HCC codes) provided in the medical condition fields 108. The value in the risk adjustment factor score field 104 may differ depending on the risk strategy, which may be toggled among plural risk strategies in the navigate/features bar 102. The suspect over-coding fields 110 are those the validation module 84 have determined to be possible over-coded conditions, and may include a recommendation (Y or N) and a confidence measure (e.g., percentage) as similarly shown in FIG. 2. The suspect missing codes field 112 is similarly configured, and includes those HCC codes that the validation module 84 has determined to be possibly missing.

Having described various embodiments of an HCC coding notification system, it should be appreciated that one HCC coding notification method, denoted as method 114, comprises receiving information from plural patients, the information from each of the plural patients comprising one or more items recorded over a predefined period of time (116) and formatting the items for use in a machine learning algorithm (118). In one embodiment, the formatting (118) comprises associating each of the one or more items with a respective word (120); forming a sentence for each of the plural patients from the words (122); and transforming the sentences for the plural patients into a numerical matrix, wherein the matrix comprises one row per patient and one column per word, each word weighted by occurrence (124). The method 114 further comprises training models according to the machine learning algorithm based on the formatted items of the plural patients (126); determining if a first patient should have a hierarchical condition category code by applying one or more formatted items of the first patient to the trained models (128); and providing a notification of any suspect hierarchical condition category codes based on the application of the one or more formatted items of the first patient to the trained models (130).

Any process descriptions or blocks in the flow diagram illustrated in conjunction with the present description should be understood as representing data, modules, segments, or portions of code which include one or more executable instructions for implementing specific logical functions or steps in the process, and alternate implementations are included within the scope of an embodiment of the present invention in which functions may be executed substantially concurrently and/or in a different order, and/or additional logical functions or steps may be added, depending on the functionality involved, as would be understood by those reasonably skilled in the art of the present invention.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. For instance, though the discussion focuses on the training and validation based on items that include cares, medications, and labs, some embodiments may use fewer items (e.g., cares only, cares and medications only, etc.) and some embodiments may use additional embodiments (e.g., vitals). It should be appreciated within the context of the present disclosure that models trained with more patient domains (e.g., more items) can achieve better performance than those with fewer domains. For instance, experimentation indicates that adding medications to care may significantly improve performance when compared to the use of cares alone. Similarly, adding labs to the cares and medications can improve performance. Using fewer domains may improve speed of performance and/or reduce processing requirements. Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Any reference signs in the claims should be not construed as limiting the scope.

1. A computer-implemented method, comprising:
receiving information from plural patients, the information from each of the plural patients comprising one or more items recorded over a predefined period of time;
formatting the items for use in a machine learning algorithm, wherein the formatting comprises:
associating each of the one or more items with a respective word;
forming a sentence for each of the plural patients from the words; and
transforming the sentences for the plural patients into a numerical matrix, wherein the matrix comprises one row per patient and one column per word, each word weighted by occurrence;
training models according to the machine learning algorithm based on the formatted items of the plural patients;
determining if a first patient should have a hierarchical condition category code by applying one or more formatted items of the first patient to the trained models; and
providing a notification of any suspect hierarchical condition category codes based on the application of the one or more formatted items of the first patient to the trained models.

2. The method of claim 1, wherein the formatting comprises quantifying an importance of each of the one or more items of the plural patients.

3. The method of claim 1, wherein the training comprises building a plurality of independent decision trees from the matrix, wherein each decision tree is built based on ordering nodes of each of the decision trees according to the importance of each word to the suspect hierarchical category code.

4. The method of claim 3, wherein the formatted items of the first patient is achieved by:
associating each of the items of the first patient with a respective word;
forming a sentence for the first patient from the words; and
transforming the sentence into a numerical matrix.

5. The method of claim 4, wherein the applying comprises:
passing the words of the transformed sentences through all of the plurality of decision trees; and
tallying outcomes from all of the plurality of decision trees, wherein the determining is based on the tallied outcomes for each of the words passed through all of the plurality of decision trees.

6. The method of claim 3, wherein building includes building hierarchical condition category code models according to a training phase, wherein each model corresponds uniquely to one of the hierarchical condition category code models.

7. The method of claim 6, wherein each outcome is based on applying the words of the patient to all of the hierarchical condition category code models.

8. The method of claim 1, wherein providing the notification comprises recommending to a user that a hierarchical condition category code recorded for the first patient is possibly over-coded.

9. The method of claim 8, wherein the notification includes a confidence measure.

10. The method of claim 1, wherein providing the notification comprises recommending to a user that a hierarchical condition category code for the patient is possibly missing.

11. The method of claim 10, wherein the notification includes a confidence measure.

12. The method of claim 1, wherein the one or more items includes one or any combination of cares, medications, labs, or vitals.

13. The method of claim 1, wherein transforming includes tokenizing the sentences with word separators, counting occurrences of each of the words in each of the sentences, and normalizing and weighting with diminishing importance the words that occur in a majority of the patients.

14. A system, comprising:
one or more computing devices configured to:
receive information from plural patients, the information from each of the plural patients comprising one or more items recorded over a predefined period of time;
format the items for use in a machine learning algorithm, wherein the formatting comprises:
associating each of the one or more items with a respective word;
forming a sentence for each of the plural patients from the words; and
transforming the sentences for the plural patients into a numerical matrix, wherein the matrix comprises one row per patient and one column per word, each word weighted by occurrence;
train models according to the machine learning algorithm based on the formatted items of the plural patients;
determine if a first patient should have a hierarchical condition category code by applying one or more formatted items of the first patient to the trained models; and
provide a notification of any suspect hierarchical condition category codes based on the application of the one or more formatted items of the first patient to the trained models.

15-19. (canceled)

20. A non-transitory computer readable medium encoded with instructions executable by a processor or processors that causes the processor or processors to:
receive information from plural patients, the information from each of the plural patients comprising one or more items recorded over a predefined period of time;
format the items for use in a machine learning algorithm, wherein the formatting comprises:
associating each of the one or more items with a respective word;
forming a sentence for each of the plural patients from the words; and
transforming the sentences for the plural patients into a numerical matrix,
wherein the matrix comprises one row per patient and one column per word, each word weighted by occurrence;
train models according to the machine learning algorithm based on the formatted items of the plural patients;
determine if a first patient should have a hierarchical condition category code by applying one or more formatted items of the first patient to the trained models; and
provide a notification of any suspect hierarchical condition category codes based on the application of the one or more formatted items of the first patient to the trained models.
